(19) United States
(12) Patent Application Publication
(10) Pub. No.: US 2002/0004204 A1
(43) Pub. Date: Jan. 10, 2002

(54) Microarray substrate with integrated photodetector and methods of use thereof

(76) Inventor: Matthew T. O'Keefe, Saratoga State, CA (US)

Correspondence Address: Paula A. Borden, BOZICEVIC, FIELD & FRANCIS LLP, 200 Middlefield Road, Suite 200, Menlo Park, CA, 94025, US

(21) Appl. No.: 09/796,932

(22) Filed: Feb. 28, 2001

Related U.S. Application Data
(60) Provisional application No. 60/185,878, filed on Feb. 29, 2000.

(51) Int. Cl. C12Q 1/68

(52) U.S. Cl. 435/6; 435/287.2

(57) ABSTRACT

The present invention provides a microarray substrate comprising a plurality of photodetectors integrated therein. The invention further provides a detection device for use in conjunction with a microarray substrate of the invention, as well as methods of use of same.

### FIELD OF THE INVENTION

This invention is in the field of biopolymer microarrays, and in particular, microarray substrates and devices for detecting emitted light signals from biopolymer microarrays.

### BACKGROUND OF THE INVENTION

Arrays of binding agents, such as oligonucleotides, have become an increasingly important tool in the biotechnology industry and related fields. These arrays, in which a plurality of binding agents are deposited onto a solid support surface in the form of an array or pattern, find use in a variety of applications, including drug screening, nucleic acid sequencing, mutation analysis, and the like. One important use of arrays is in the analysis of differential gene expression, where the expression of genes in different cells, normally a cell of interest and a control, is compared and any discrepancies in expression are identified. In such assays, the presence of discrepancies indicates a difference in the classes of genes expressed in the cells being compared.

Microarrays of biopolymers are now in wide use for a variety of purposes. For example, microarrays of DNA are used in applications such as sequencing a nucleic acid molecule; fingerprinting, e.g., in application such as forensics; mapping a nucleic acid molecule; screening for polymorphisms; and determining expression patterns.

The biopolymer is labeled, directly or indirectly, with a detectable label. Among the most commonly used labels are fluorescers, chemiluminescers, chromogenic labels, and spectroscopic labels. Among these, fluorescent labels are in wide use.

Devices for detecting fluorescently marked targets on devices are known in the art. Generally, such detection devices include a microscope and light source for directing light at a substrate. See, for example, U.S. Pat. No. 5,143,854; and published International Patent Application No. WO 92/10092. A photon counter detects fluorescence from the substrate, while an x-y translation stage varies the location of the substrate. An example of a detection device used to scan the microarray is a confocal detection device, such as those described in U.S. Pat. Nos. 5,631,734; and 5,091,652. A scanning laser microscope is described in Shalon et al. (1996) Genome Res. 6:639. A scan, using the appropriate excitation line, is performed for each fluorophore used. The digital images generated from the scan are then combined for subsequent analysis. These devices are large, and the cost of such devices is high. These features make currently available devices unfeasible for general use, e.g., in a clinical or general research laboratory setting.

The foregoing discussion illustrates the need in the art for a more compact, easily manufactured device for detecting labeled biopolymer targets immobilized on microarrays. The present invention addresses this need and provides related advantages as well.

### SUMMARY OF THE INVENTION

The present invention provides a microarray solid substrate, such as a slide or a wafer, which comprises integrated detectors of radiant energy. A microarray solid substrate of the invention generally comprises a photodetector integrated into the solid substrate; a polymer sequence microarray, located directly above an integrated photodetector and on a first surface of the light detector; and an integrated signal transmission means, which signal transmission means is in direct contact with an integrated photodetector. In some embodiments, the photodetector is a photodiode. In some embodiments the microarray substrate is a slide and is made of a material comprising silicon. In some of these embodiments, the microarray slide further comprises a plurality of positionally distinguishable polymer sequences arranged in spots directly above each photodetector in the solid substrate, i.e., a given spot is in register with a given photodetector. In particular embodiments, the biopolymer is a polynucleotide.

In some embodiments, the substrate comprises a first layer and a second layer. The first layer is referred to as the polymer layer and comprises a plurality of positionally distinguishable polymer sequences arranged in spots. The second layer is referred to as the photodetector layer and comprises a photodetector and signal transmission means. The first layer may be detachably positioned on the second layer such that the first layer can be removed from the second layer.

In use, a biopolymer is labeled, directly or indirectly, with a moiety which emits radiant energy, e.g., light. An integrated photodetector which is positioned underneath the microarray spot detects an emitted light signal, and generates an electrical signal corresponding to the intensity of the detected light. Output from the photodetector is transmitted to a reading device by a signal transmission means such as an electrically conducting material integrated into the slide. In some embodiments, each photodetector comprises positional address information.

In some embodiments, the present invention provides a device comprising a substrate which provides a surface; a plurality of different probe polymer sequences bound to the surface, wherein each different sequence is bound to a distinct area of the surface; a plurality of photodetectors positioned in a manner such that a signal emitted from a distinct area of probe polymer sequences can be detected and differentiated from a signal of another distinct area of probe polymer sequences.

The present invention further provides a device for detecting and processing an electronic signal from a microarray solid substrate of the invention. The device comprises a body or stage for immobilizing the substrate; and a reading device for reading a signal from the signal transmission means. The device may further comprise a microprocessor for storing, managing, and processing information provided by electronic signals detected by the reading means. In some embodiments, the device is adapted for detecting fluorescently-labeled materials on the microarray, and comprises a monochromatic or polychromatic light source; a means for directing an excitation light from the light source onto the microarray solid substrate; a means for focusing the light onto the substrate; a detection means for detecting a signal transmitted from a photodetector integrated into the substrate; and a means for identifying the region from which the signal originated. The means for focusing the excitation light onto a point on the substrate and determining the region from which the detected signal originated may include an x-y translation table. The device may further comprise a means for controlling temperature of the substrate during, e.g., a binding reaction. In additional embodiments, translation of the x-y table, and data collection are recorded and processed by an appropriately programmed digital computer.

The invention further provides a method of detecting a binding agent in a microarray, generally comprising contacting a labeled polymer with a polymer immobilized on a substrate as described in the present invention; introducing the substrate into a detection device, whereby a signal generated by a labeled polymer bound to a polymer is detected by the detection device. In some embodiments, the method comprises allowing a labeled target molecule to hybridize to a probe molecule bound to a substrate, forming a probe-target hybrid; and detecting a signal from the probe-target hybrid using a photodetector positioned adjacent the probe molecule.

An advantage of the microarray substrate of the invention is that each photodetector can be addressable, allowing identification of the signal-generating binding agent.

A further advantage of the microarray substrate of the invention is that integration of photodetectors into the substrate reduces or eliminates “cross-talk,” i.e., detection of radiant energy (e.g., light) from adjacent microarray regions with which the photodetector is not in register is reduced or eliminated. This feature allows microarray spots to be provided in the microarray substrate at high density.

A further advantage of the microarray substrate of the invention is that, in those embodiments in which the substrate comprises a first (polymer) layer and a second (photodetector) layer, the first layer comprising bound probe biopolymer sequences can be physically removed from the second layer comprising the photodetector and signal transmission means. Thus, the second layer can be re-used multiple times with different first layers.

A further advantage of the microarray substrate of the invention is that the distance between a microarray and a photodetector is extremely small, and as a consequence, light collection efficiency is greatly improved, and signal to noise ratio is significantly enhanced.

A further advantage of the microarray substrate of the invention is that a lower limit of detection is achieved.

A feature of the invention is that currently available photodiode arrays available in devices such as cameras can be used as substrate for the biopolymer arrays of the present invention.

These and other objects, advantages, and features of the invention will become apparent to those persons skilled in the art upon reading the details of the invention as more fully described below.

### BRIEF DESCRIPTION OF THE DRAWINGS

FIGS. 1 and 2 depict various views of an exemplary embodiment of a microarray substrate of the invention.

FIG. 1 is a cut-away view;

FIG. 2 is a perspective view of a first surface.

FIG. 3 depicts a further exemplary embodiment of a microarray substrate of the invention, and shows a microarray substrate layer which is removable from a photodiode substrate layer.

FIGS. 4A and 4B depict a further exemplary embodiment of a microarray substrate of the invention, and shows a substrate comprising louvers as a signal selection means.

FIG. 5 depicts an exemplary embodiment of a detection device of the invention.

FIG. 6 depicts a further exemplary embodiment of a detection device of the invention.

### MODES OF CARRYING OUT THE INVENTION

The arrays of the present invention comprise: (1) a substrate surface having a plurality of photodetectors; and (2) polymer sequences attached to the surface in a manner which allows detection of an individual spot or defined area of identical sequences. The photodetector is any element that is capable of detecting light and converting it into an electrical signal. The surface may have any desired shape but is preferably planar. The biopolymer may be any type of polymer capable of providing information, but is preferably a sequence of nucleotides. The array may be comprised of any number of photodetectors over any desired area.

It is possible to associate a plurality of spots or distinct areas of polymer sequences with a single photodetector or to associate a plurality of photodetectors with a single spot or distinct area. However, the simplest arrangement is to associate a single photodetector with a single spot or distinct area of polymer sequences, i.e., a single photodetector is in register with a single spot of polymer sequence. Further, the photodetectors are preferably designed such that they are not receiving significant interference from surrounding signals.

The array may be constructed in a variety of different configurations and the simplest is to bind polymer sequences directly to the photodetector. However, it is possible to include a protective layer or substrate over the photodetector and to attach the sequences to the protecting layer. The invention can be designed as a system wherein the protecting layer (i.e., a microarray substrate layer which holds the polymer sequences) is removable from the photodetectors (i.e., from a photodetector substrate comprising the photodetectors) positioned underneath. A number of different removable protecting layers can be part of a system which can be designed to allow the layers to be quickly moved into and out of position. Using such a system, one can quickly obtain information from a large number of different sequence arrays and/or from duplicate sequence arrays which have been used to test different samples.

In each embodiment, the photodetector detects a light signal emitted from a polymer array and generates an electrical signal corresponding to the intensity of the detected light. This electrical signal is then transmitted to a reading device by the signal transmission means such as an electrically conducting material.

In general, a microarray substrate comprises a microarray on a first planar surface of the substrate; a photodetector integrated into the substrate just below the microarray and extending partially through the thickness of the substrate. In these embodiments, the signal transmission means is integrated within the substrate, and may interdigitate among the photodetectors. The signal transmission means may further comprise a signal amplification means, and/or may further comprise a switch means. Integrated circuitry which is well-known in the art can be used as the signal transmission means.

The microarray substrate and reading device confer a number of advantages over currently available devices for detecting binding agents such as polymers. Since the photodetectors are integrated into the microarray substrate, the substrate, and consequently the reading device, can be significantly smaller and more compact than currently available devices. In addition, since the photodetectors are integrated into the substrate, they are in close physical proximity to the polymers, and hence to any emitted signals from signal-emitting moieties associated with a polymer. This close physical association results in greater sensitivity of detection, and greater signal to noise ratio.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a microarray” includes a plurality of such microarrays and reference to “the device” includes reference to one or more devices and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

The term “microarray substrate,” as used herein, refer to a substrate having a plurality of biopolymers stably attached to its surface, where the biopolymers may be spatially located across the surface of the substrate in any of a number of different patterns.

The term “integrated photodetector” and “integrated signal transmission means,” as used herein, refers to photodetectors and signal transmission means, respectively, which are embedded wholly or partially within the microarray substrate. Embedding or integrating is generally accomplished using microfabrication and microlithography techniques known in the art.

The terms “polymer”, “biopolymer”, “sequence(s)”, and the like, are used interchangeably herein to refer to any substance, typically a polymer, that is specifically recognized by another substance, also typically a polymer, i.e., is a member of a specific binding pair, where such specific binding pairs include: peptides, e.g. proteins or fragments thereof, binding to antibodies; nucleic acids, e.g. oligonucleotides, polynucleotides binding to complementary nucleic acids; sugars, oligosaccharides, and polysaccharides binding to lectins; ligands, agonists, and antagonists binding to a polypeptide or glycoprotein receptor; enzyme substrates, cofactors, and inhibitors binding to enzymes; and the like. Polymers include biopolymers (e.g., polynucleotides, oligonucleotides, polypeptides, etc.). Any given polymer may be in solution, or may be associated with (i.e., bound to the surface of) the microarray substrate. Polymers include naturally-occurring compounds, modifications of such compounds, synthetic compounds, and semi-synthetic compounds. Polymer sequences may be directly bound to a substrate surface or connected via a linker, or binding agent, a variety of which are known in the art.

The term “polypeptide” refers to a polymer of amino acids and does not refer to a specific length of the product; thus, peptides, oligopeptides, and proteins are included within the definition of polypeptide. The term includes modified polypeptides, including, but not limited to post-translational modifications of the polypeptide, for example, glycosylations, acetylations, phosphorylations and the like. Included within the definition are, for example, polypeptides containing one or more analogs of an amino acid (including, for example, unnatural amino acids, non-coded amino acids, etc.), polypeptides with substituted linkages, as well as other modifications known in the art, both naturally occurring and non-naturally occurring.

The terms “polynucleotide” and “nucleic acid”, used interchangeably herein, refer to a polymeric forms of nucleotides of any length, either ribonucleotides or deoxynucleotides. The sequence is preferably four or more, and more preferably six or more, nucleotides in length. Lengths of six to 18 are preferred in some embodiments. In other embodiments, longer polynucleotides are used, e.g., 20, 25, 30, 35, 40, 45, or 50 nucleotides in length. The term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. Single-stranded sequences are preferred. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidites and thus can be an oligodeoxynucleoside phosphoramidate or a mixed phosphoramidate-phosphodiester oligomer. Peyrottes et al. (1996) Nucl. Acids Res. 24:1841-1848; Chaturvedi et al. (1996) Nucl. Acids Res. 24:2318-2323. A polynucleotide may comprise modified nucleotides, such as methylated nucleotides and nucleotide analogs, uracyl, other sugars, and linking groups such as fluororibose and thioate, and nucleotide branches. Arrays of modified nucleotide sequences are taught in European Patent No. EP 742,287. The sequence of nucleotides may be interrupted by non-nucleotide components. A polynucleotide may be further modified after polymerization, such as by conjugation with a labeling component. Other types of modifications included in this definition are caps, substitution of one or more of the naturally occurring nucleotides with an analog, and introduction of means for attaching the polynucleotide to proteins, metal ions, labeling components, other polynucleotides, or a solid support.

The term “hybridization,” in the context of polynucleotide-polynucleotide interactions, is a term well known in the art and refers to the association of two nucleic acid sequences to one another by hydrogen bonding, usually on opposite nucleic acid strands (i.e., two strands of opposite polarity), or two regions of a single nucleic acid strand. Guanine and cytosine are examples of complentary bases, which are known to form three hydrogen bonds between them. Adenine and thymine are examples of complementary bases which form two hydrogen bonds between them. “Hybridization” refers to the association of two nucleic acid sequences to one another by hydrogen bonding. Two sequences will be placed in contact with one another under conditions that favor hydrogen bonding. Factors that affect this bonding include: the type and volume of solvent; reaction temperature; time of hybridization; agitation; agents to block the nonspecific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook, et al., Molecular Cloning: A Laboratory Manual, 2nd Ed. (1989), Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 12° C. to 20° C. below the calculated Tₘ of the hybrid under study. The temperature and salt conditions can often be determined empirically in preliminary experiments in which samples of genomic DNA immobilized on filters are hybridized to the sequence of interest and then washed under conditions of different stringencies. See Sambrook, et al., supra, at page 9.50.

Several factors can affect the melting temperature (Tₘ) of a DNA-DNA hybrid between the target and sequence of interest, and consequently, the appropriate conditions for hybridization and washing. In many cases the target is not 100% homologous to the fragment. Other commonly encountered variables include the length and total G+C content of the hybridizing sequences and the ionic strength and formamide content of the hybridization buffer. The effects of all of these factors can be approximated by a single equation:

Tₘ=81+16.6(log10Ci)+0.4[%G+C)]−0.6(%formamide)−600/n−1.5(%mismatch),

where Ci is the salt concentration (monovalent ions) and n is the length of the hybrid in base pairs (slightly modified from Meinkoth and Wahl (1984) Anal. Biochem. 138:267-284).

Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10× SSC, 6× SSC, 1× SSC, 0.1× SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6× SSC, 1× SSC, 0.1× SSC, or deionized water. One non-limiting example of stringent conditions are hybridization and washing at 50° C. or higher and in 0.1× SSC (9 mM NaCl/0.9 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Stringent hybridization conditions are hybridization conditions that are at least as stringent as the above representative conditions. Other stringent hybridization conditions are known in the art and may also be employed to identify nucleic acids of this particular embodiment of the invention.

For hybridization probes, it may be desirable to use nucleic acid analogs, in order to improve the stability and binding affinity. See, e.g., EP 742,287. A number of modifications have been described that alter the chemistry of the phosphodiester backbone, sugars or heterocyclic bases.

Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH₂-5′-O-phosphonate and 3′-NH-5′-O-phosphoroamidate. Peptide nucleic acids replace the entire phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5- propynyl-2′-deoxyuridine and 5-propynyl-2′-deoxycytidine have been shown to increase affinity and biological activity when substituted for deoxythymidine and deoxycytidine, respectively.

The terms “radiation” and “radiant energy,” used interchangeably herein, refer to energy which may be selectively applied to, and/or which is emitted from, a microarray substrate of the invention, and includes energy having a wavelength of between 10⁻¹⁴ and 10⁴ meters, including, e.g., electron beam radiation, gamma radiation, x-ray radiation, ultraviolet radiation, visible light, infrared radiation, microwave radiation, and radio waves. “Irradiation” refers to the application of radiation to a surface.

Biopolymer Substrate Materials and Characteristics

The present invention provides a microarray substrate. In some embodiments, the microarray substrate comprises (a) a plurality of distinct spots or regions, each spot or region comprising a plurality of substantially identical polymers stably associated with a first planar surface of a solid substrate; (b) a plurality of photodetectors integrated into said solid substrate and extending partially through a thickness of said solid substrate, wherein a photodiode is positioned directly beneath a spot or region comprising a plurality of substantially identical polymers; and (c) a signal transmission means integrated in the microarray substrate, which provide for transmission of an electronic signal generated by a photodetector to a reading device.

In other embodiments, the microarray substrate is provided in at least two sections or layers: a first, polymer layer; and a second, photodetector layer. The first layer comprises the microarray spots, and is physically separable from the second layer, which comprises the integrated photodetectors and the integrated signal transmission means. The polymer layer is sometimes referred to herein as a “protective layer.” The polymer layer can be detachable (i.e., removable) from the photodetector layer. In these embodiments, the photodetector layer can be reused multiple times with different polymer layers. The photodetector layer may be connected to the polymer layer in any of a variety of ways. As non-limiting examples, the photodetector layer may have pegs arranged at the corners, which fit into holes at analogous positions in the polymer layer; there may be complementary protrusions/slots in the two layers; the two layers may be clipped together by removable clips; the microarray substrate layer may simply be placed on top of the photodetector substrate layer; and the like.

The microarray substrate employed in the subject invention may be any convenient configuration, but generally has a planar configuration. By “planar configuration” is meant that the substrate has at least one planar surface, which surface may have any convenient cross-sectional shape, including circular, oval, square, rectangular and the like. In many embodiments, the substrate has a plate-like configuration, such as is found in a disk, rectangular slide, square slide, and the like. The substrate may contain raised or depressed regions on which a polymer sample is located. The substrate generally provides a rigid support on which the polymer sample is located. The polymer sample is located on a first surface of the substrate.

In many embodiments in which the ultimate array is to have a planar configuration, the substrate comprises at least one planar surface that has a surface area of at least about 4 mm², usually at least about 16 mm² and more usually at least about 25 mm², where the cross-sectional area of the planar surface may be as large as 2500 mm² or larger, but generally does not exceed about 900 mm² and usually does not exceed about 400 mm². In those embodiments where the planar surface has a square or rectangular shape, the planar surface has a length of from about 2 to 50 mm, usually from about 4 to 30 mm and more usually from about 5 to 20 mm, and has a width ranging from about 2 to 50 mm, usually from about 4 to 30 mm and more usually from about 5 to 20 mm. The substrate thickness may vary considerably, depending on the detection protocol, i.e. whether detection is through the substrate or just on the surface. For example, where the array is to be read through the substrate, the thickness generally ranges from about 0.7 to 1.2 mm. Alternatively, where the array is to be surface read, the thickness is generally dictated by the substrate fabrication process.

The substrate may comprise functionalized glass; glass, e.g., SiOₓ, borosilicate; Si, SiO₂, SiN₄, modified silicon; Ge, GaAs; or any of a wide variety of gels or polymers, including, but not limited to, polytetrafluoroethylene, polyvinylidene difluoride, polystyrene, polycarbonate, and combinations thereof. In some embodiments, the substrate is silica or glass. Where the substrate comprises silicon, the silicon need not be pure silicon, but may be semiconductor-grade silicon. Where the substrate is silicon or another glass, the material is typically derivatized.

Very Large Scale Immobilized Polymer Synthesis (VLSIPS™) methods of producing large arrays of biopolymers are well known in the art and can be used in the present invention. For example, methods of producing large arrays of oligopeptides and oligonucleotides are described in U.S. Pat. No. 5,134,854 (Pirrung et al.), and U.S. Pat. No. 5,445,934 (Fodor et al.) using light-directed synthesis techniques. Using a computer controlled system, a heterogeneous array of monomers is converted, through simultaneous coupling at a number of reaction sites, into a heterogeneous array of polymers. Alternatively, microarrays are generated by deposition of presynthesized oligonucleotides onto a solid substrate, for example as described in International Patent application WO 95/35505.

DNA arrays may be prepared manually by spotting DNA onto the surface of a substrate with a micro pipette. See Khrapko et al.(1991) DNA Sequence 1:375-388. Alternatively, the dot-blot approach, as well as the derivative slot-blot approach, may be employed in which a vacuum manifold transfers aqueous DNA samples from a plurality of wells to a substrate surface. In yet another method of producing arrays of biopolymeric molecules, a pin is dipped into a fluid sample of the biopolymeric compound and then contacted with the substrate surface. By using a plurality or array of pins, one can transfer a plurality of samples to the substrate surface at the same time. Alternatively, an array of capillaries can be used to produce biopolymeric arrays. See WO 95/35505. In another method of producing biopolymeric arrays, arrays of biopolymeric agents are “grown” on the surface of a substrate in discrete regions. See e.g. U.S. Pat. No. 5,143,854; and Fodor et al. (1991) Science 251:767-773.

Sequences on the substrate are referred to as “probe sequences” and the sequences that they bind to are referred to as “target sequences.” Arrays with a probe density as high as 400 or more oligonucleotides per cm² have been described by others (see, e.g., U.S. Pat. No. 5,744,305, issued Apr. 28, 1998). Others have described arrays with probe densities of as high as 1,000 or more nucleotides per cm² (see, e.g., U.S. Pat. No. 5,445,934, issued Aug. 29, 1995).

Generally, the structures onto which the fluid sample is deposited in the subject microarray substrates comprise a substrate surface having at least one location thereon occupied by a composition made up of a single type of polymer, e.g. identical proteins, nucleic acids with the same sequence, etc., where this homogenous composition is present on the substrate surface in the form a spot or some other shape. In many embodiments, the subject substrates are employed to deposit a volume of fluid sample onto the surface of an array. Arrays onto which fluid sample is deposited in the subject substrates are compositions of matter having a plurality of distinct polymers, e.g. single-stranded nucleotide probes, stably associated with a substrate surface, where the plurality of polymers is generally known and positioned across the surface of the array in a pattern. Each distinct polymer present on the array is generally a member of a specific binding pair. Polymers of interest are generally biological molecules or biomolecules and include: polypeptides, nucleic acids, carbohydrates, glycoproteins, etc. As such, binding pairs in which one member thereof is stably associated to the array surface include: ligands and receptors; antibodies and antigens; complementary nucleic acids; etc. As mentioned above, the plurality of polymers are arranged across the surface of a substrate in the arrays. Typically, the arrays comprise a plurality of spots, where each spot contains a different and distinct polymer, i.e. the arrays comprise a plurality of homogenous polymer compositions, where each composition is in the form of a spot on the substrate surface of the array.

The number of spots on a substrate surface in any given array varies greatly, where the number of spots is at least about 1, usually at least about 10 and more usually at least about 100, and may be as great as 100,000 or greater, but usually does not exceed about 10⁷ and more usually does not exceed about 10⁶. The spots may range in size from about 0.1 μm to 10 mm, usually from about 1 to 1000 μm and more usually from about 10 to 100 μm. The density of the spots may also vary, where the density is generally at least about 1 spot/cm², usually at least about 100 spots/cm² and more usually at least about 400 spots/cm², where the density may be as high as 10⁶ spots/cm² or higher, but generally does not exceed about 10⁵ spots/cm² and usually does not exceed about 10⁴ spots/cm². A variety of arrays are known to those of skill in the art, where representative arrays include those disclosed or referenced in: U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,624,711; 5,639,603; 5,658,734; as well as in WO 93/17126; WO 95/11995; WO 95/35505; EP 742 287; and EP 799 897; the disclosures of which are herein incorporated by reference. Of particular interest in many embodiments of the subject methods is the deposition of a fluid sample onto arrays of nucleic acids, including arrays of oligonucleotides and polynucleotides, e.g. cDNAs.

Surfaces on the solid substrate will usually, though not always, be composed of the same material as the substrate. Alternatively, the surface may be composed of any of a wide variety of materials, for example, polymers, plastics, resins, polysaccharides, silica or silica-based materials, carbon, metals, inorganic glasses, membranes, or any of the above-listed substrate materials. In some embodiments the surface may provide for the use of caged binding members which are attached firmly to the surface of the substrate. Preferably, the surface will contain reactive groups, which could be carboxyl, amino, hydroxyl, or the like. Most preferably, the surface will be optically transparent and will have surface Si—OH functionalities, such as are found on silica surfaces.

The signal transmission means is integrated within the microarray substrate, or, in those embodiments in which the microarray substrate comprises two or more layers, is integrated within the photodetector layer. The signal transmission means generally comprises an electrically conducting material, a variety of which are known in the art. The signal transmission means may interdigitate among the photodetectors. The signal transmission means may further comprise a signal amplification means, and/or may further comprise a switch means. Integrated circuitry which is well known in the art can be used as the signal transmission means. Integration of the signal transmission means within the substrate can be accomplished by any of a variety of known microfabrication techniques. The signal transmission means is in operable linkage, i.e., is operably connected to, the photodetector(s), i.e., the signal transmission means is capable of transmitting the signal generated by the photodiode in response to radiant energy to a reading device. The signal transmission means may be in direct contact with a photodetector, but need not be. Thus, in some embodiments, the signal transmission means is not in direct contact with a photodetector, but is in physical proximation to a photodetector such that a signal emitted from a photodetector is detected by the signal transmission means.

A microarray may further comprise a means for regulating temperature of the substrate. Means for regulating the temperature of the substrate may be embedded within the substrate, or may be positioned on the second planar surface (i.e., the surface opposite the surface on which the polymers are located). Regulating the temperature may find use in applications in which association of complementary polymers is affected by temperature. As an example, stringency of nucleic acid hybridization is affected, in part, by temperature. As one non-limiting example, the temperature may be increased to 68° C. for stringent nucleic acid hybridization conditions. Nucleic acid hybridization conditions have been described above. Regulating the temperature may also find use in enzymatic reactions, where the temperature is adjusted to the temperature optimum of the enzyme being used. As an example, a reaction using an enzyme derived from an extreme thermophile can be carried out. A non-limiting example of such reactions is a polymerase chain reaction using a thermostable DNA polymerase (e.g., from Thermus aquaticus). Alternatively, the temperature can be adjusted so as to inactivate an enzyme, e.g., by raising the temperature well above the temperature optimum for an enzyme.

The means for regulating temperature can also be one that cools the substrate to temperatures below about −10° C., below about −20° C., or below about −30° C., down to about −40° C. Cooling the substrate to such low temperatures once the binding/hybridization reaction has already occurred confers the advantage of further reducing electrical noise, i.e., cross-talk, i.e., electrical signals from neighboring microarray spots, or other extraneous electrical signals.

Thus, a temperature regulator can regulate the temperature in a range of from about −40° C. to about 95° C., from about −30° C. to about 90° C., from about −20° C. to about 80° C., from about −10° C. to about 75° C., from about 0° C. to about 65° C., from about 4° C. to about 60° C., from about 10° C. to about 50° C., from about 17° C. to about 45° C., or from about 25° C. to about 30° C., or any selected temperature or temperature range within any of the foregoing ranges. In addition, a temperature regulator may provide for, e.g., a progressive increase or decrease in temperature over time, or may provide for a cycle(s) of two or three different temperatures (e.g., 95° C., 50° C., 72° C.).

Integrated Photodetectors

Integrated into the substrate are one or more, usually a plurality of, photodetectors. The photodetectors convert a detected radiant energy signal into an electrical signal. Each photodetector is aligned with (i.e., in register with) a microarray, and positioned underneath each microarray. A photodetector has a first end, which is proximal to the microarray, and a second end, which is distal to the microarray and which extends partially through the thickness of the substrate. The photodetector is in contact with a signal transmission means, such as an electrically conductive material, which transmits an electrical signal to a detection means. The signal transmission means may be an electrically conductive means or material.

The photodetectors are generally present in a density of from about 10 to about 100, from about 100 to about 500, from about 500 to about 1000, from about 1000 to about 5000, from about 5000 to about 10⁵, from about 10⁵ to about 5×10⁵, from about 5×10⁵ to about 10⁶, up to about 10⁷ per square centimeter of surface area. The spacing between photodetectors, e.g., the inter-photodetector distance not occupied by a photodetector can be from about 1 nm to about 5 mm, from about 10 nm to about 1 mm, from about 100 nm to about 10⁵ μm, from about 1 μm to about 10⁴ μm, or from about 100 μm to about 1000 μm.

In general, the second end of the photodetector extends only partially through the thickness of the substrate or substrate layer. Generally, the signal transmission means is integrated (e.g., embedded) within the substrate, e.g., the signal transmission means could interdigitate between and among the photodetectors. Standard integrated circuitry well-known in the art may be used. The signal transmission means may further comprise a signal amplification means, and/or a switch means.

Photodetectors suitable for use in a microarray substrate of the invention include any element which is capable of detecting radiant energy and converting the detected radiant energy into an electrical signal. Suitable photodetectors include, but are not limited to, photodiodes, charge-coupled devices (CCDs), photoconductive cells, avalanche photodiodes, photoresistors, photoswitches, phototransistors, phototubes, photovoltaic cells, light-to-frequency converters, or any other type of photosensor capable of converting light into an electrical signal. Such photodetectors can include integrated conversion of light to voltage with electronic amplification components; integrated conversion of light to digital frequency components; or integrated analog to digital conversion components.

In general, a photodiode may comprise functionalized glass; glass, e.g., SiOₓ, borosilicate; Si, SiO₂, SiN₄, modified silicon; Ge, GaAs; and may be coated with any of a wide variety of gels or polymers, including, but not limited to, polytetrafluoroethylene, polyvinylidene difluoride, polystyrene, polycarbonate, and combinations thereof. In some embodiments, the photodiode is comprised of a silicon or a glass.

In some embodiments, photodetectors are arranged in ordered arrays, aligned with members of a biopolymer microarray. A photodetector is positioned just underneath a microarray, generally at a distance of between about 0.01 μm and about 100 μm, between about 0.05 μm and about 50 μm, or between about 0.1 μm and about 10 μm This distance may be varied, depending on several factors, including, e.g., the thickness of the filtering, or passivating layer, as discussed below. In some embodiments, the polymers may be attached directly to the photodetector.

The extremely short distance between the polymer and the photodetector confers an advantage in that it enhances the efficiency of light collection, and minimizes detection of extraneous light, e.g., from neighboring microarrays not in register with that photodetector. A plurality of photodetectors may be arranged in the substrate such that a photodetector is beneath (i.e., in register with) a spot in the microarray.

Methods for making solid substrates having photodetectors integrated therein are well known in the art, and can be used in the manufacture of the substrates of the invention. Monolithic microfabrication processes which are well known in the art may be used. As an example, U.S. Pat. No. 5,141,878 provides a description of manufacture of photodetectors within a solid substrate; U.S. Pat. No. 6,018,169 describes charge coupled device (CCD) arrays; and U.S. Pat. No. 4,903,103 describes a semiconductor photodetector device. In general, microfabrication techniques which can be used in making a microarray substrate of the invention may be found in standard textbooks, including, e.g., Micromachined Transducer Sourcebook, G. Kovacs (1998) WCB/McGraw-Hill; Physics of Semiconductor Devices, S. M. Sze (1981), John Wiley & Sons; Fundamentals of microfabrication, M. J. Madou (1997) CRC Press; Laser microfabrication: Thin film processes and photolithography, D. J. Erlich and J. Y. Tsao, eds. (1989) Academic Press; and Handbook of microlithography, micromachining and microfabrication, P. Rai-Choudhury, ed. (1997) Society of Photo-optical Instrumentation Engineers.

An integrated photodetector can be addressable so that the microspot from which the signal originated can be identified. Addressing can be achieved by any of a number of methods known in the art. In general, since each photodetector is either in direct contact, or is in close physical proximation, with a signal transmission means, the photodiode from which an electrical signal is generated can be readily determined.

Photodetectors may comprise inorganic semiconductor materials, such as silicon, which are standard in the art. Organic photodetectors have also been described and may be used in the microarray substrates of the present invention. International Patent Application Publication No. WO 99/39395.

Radiant Energy Selection Means

A microarray substrate of the invention may further comprise a means to select out undesired wavelengths of radiant energy. A radiant energy selection means is useful when a polymer is labeled with a fluorophore, and the fluorophore is excited with a laser.

When the radiant energy is generated by excitation, e.g., exciting a fluorophore with a laser, the incident light from the laser as well as the radiant energy generated by exciting the fluorophore, may be detected. Preferably, only the radiant energy generated by the fluorophore, and not the incident light from the laser, is detected. Various ways of selecting out undesired radiant energy may be employed, including, but not limited to, use of an interference filter layer; use of an optical wave guide; use of a polarization filter; time-resolved fluorescence; use of a grating, or a louver; and varying the angle of incident laser light.

A dielectric interference filter layer may be positioned on the first planar surface, between the polymer layer and the substrate layer comprising the photodetectors. The filter may comprise one or more layers of different dielectric materials of differing thicknesses to achieve an attenuation of the undesired energy wavelengths or to minimize attenuation of a desired wavelength. Such filters are known in the art and are available commercially from a variety of sources, including, e.g., ZC& R Coatings for Optics, Carlsbad, Calif. A polymer may be attached directly to the interference filter layer. The thickness of the interference filter layer can be varied, depending on the wavelength of radiant energy being filtered out. The interference filter layer may have a thickness of from about 0.01 μm to about about 100 μm, from about 0.05 μm to about 50 μm, from about 0.1 μm to about 10 μm. In addition, the interference filter layer may itself comprise more than one layer, the thickness and composition of which may be varied as needed to achieve maximal filtering out of an undesired wavelength(s).

An optical wave guide, such as an optical fiber, may be deposited on the first planar surface of the substrate. An optical wave guide guides the laser beam directly onto the polymer.

A sheet of polarizing material may be positioned between a photodetector and a polymer, forming a polarizing layer. The polarizing layer filters out the excitation light that will be polarized, and accepts only unpolarized light emitted from, e.g., a fluorophore.

When long-lived fluorophores are used, detection may be activated at a specified time after the laser light is pulsed, e.g., the photodetectors may be operably connected to a start device that delays detection for a period of time from nanoseconds to microseconds. In this way, the emission energy is differentiated from the excitation energy by a separation in time and no filtering of wavelengths is needed. For example, the photodetector can be turned on 1 μsecond after a laser pulse. For lanthanide series fluorophores, the time delay can be longer, e.g., 0.5 msecond. A single pulse, or a series of pulses, could be used, and the photodetector switched on at a pre-set time after each pulse. The photodetector could be switched on for a period of about 1 to about 100 μsecond, then switched off again before the next laser pulse.

The angle of incidence of the excitation energy source may be varied in such a way that the excitation energy does not impinge on the photodetector directly, e.g., incident at right angles to the line perpendicular to the plane of the photodetector. In this way, light emitted by the excited fluorophor may be detected by the photodetector as its emission occurs in all directions. Such a technique may be facilitated by use of a grating or louver which has been applied or deposited on the surface of the photodetector layer. Such gratings, or louvers, are known in the art, and include, but are not limited to, CRT privacy screens (3M Corp. MN). The parallel members of the grating may block or absorb radiant energy which is incident from an acute angle relative to the plane of the photodetectors. The angle beyond which excitation energy will interfere with the emission energy is the inverse tangent of the ratio of the effective height of the grating members to the effective spacing of the grating members.

The angle of incident light may be varied. The laser light can come in from the side, e.g., perpendicular to the photodetector, such that the incident light is not detected.

Uses of the Microarray Substrates of the Invention

The microarray substrates of the invention are useful in a wide variety of diagnostic methods, and other applications as well, including, e.g., manipulation and sequencing of nucleic acid samples. Diagnostic applications include, but are not limited to, diagnosing genetic disorders; detecting the presence of an infectious agent in a biological sample; forensic analyses, including but not limited to, genetic fingerprinting, identification and/or characterization of an organism, and the like.

Oligonucleotide and/or polynucleotide arrays provide a high throughput technique that can assay a large number of polynucleotides in a sample. A variety of different array formats have been developed and are known to those of skill in the art. The arrays of the subject invention find use in a variety of applications, including gene expression analysis, drug screening, mutation analysis and the like.

Applications in which a microarray substrate of the invention finds use include, but are not limited to, allele-specific oligonucleotide hybridization (Wong and Senadheera (1997) Clin. Chem. 43:1857-1861); dynamic allele-specific hybridization (DASH). Howell et al. (1999) Nat. Biotech. 17:87-88; genotyping, e.g., single nucleotide polymorphism (SNP) analysis; analysis of gene expression (e.g., differential display); enzymatic reactions, including, but not limited to, rolling circle amplification, a polymerase chain reaction, a sequencing reaction (e.g., pyrosequencing (Ronaghi (2001) Genome Res. 11:3-11), and single-base extension reactions); fluorescence resonance energy transfer (FRET) based assays; oligonucleotide ligation assays; single-base extension with fluorescence detection; homogenous solution hybridization assays (e.g., molecular beacons); Invader™ assays; time-resolved fluorescence-based assays; and the like. Many assays for genotyping are known in the art, and a microarray substrate of the invention can be used in such assays. Genotyping assays are described in, e.g., Shi (2001) Clin. Chem. 47:164-172, and references cited therein.

Arrays can be used, for example, to examine differential expression of genes and can be used to determine gene function. For example, arrays can be used to detect differential expression of a polynucleotide between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al. (1998) Sem. Radiation Oncol. 8:217; and Ramsay (1998) Nature Biotechnol. 16:40.

In some embodiments, the invention provides methods of detecting a probe molecule in a microarray, using a microarray substrate of the invention, where the target molecule is detectably labeled. These methods generally involve allowing a labeled target molecule to hybridize to a probe molecule bound to a substrate, forming a probe-target hybrid; and detecting a signal from the probe-target hybrid using a photodetector positioned adjacent the probe molecule.

In other embodiments, the invention provides methods of detecting a probe molecule in a microarray, using a microarray substrate of the invention, where a polynucleotide comprising a nucleotide sequence that is complementary to a probe molecule is synthesized and, during synthesis, becomes detectably labeled. These methods generally involve allowing an oligonucleotide primer molecule to hybridize to a probe molecule bound to a substrate, forming a probe-primer hybrid; contacting the probe-primer hybrid with a DNA polymerase, forming a reaction mixture, under conditions that promote addition of a nucleotide to the 3′ end of the primer, such that a second polynucleotide strand is generated that comprises a nucleotide sequence complementary to the probe sequence such the second polynucleotide strand hybridizes to the probe, forming a probe-second polynucleotide strand hybrid, wherein the reaction mixture comprises a labeled nucleotide, and wherein the labeled nucleotide is incorporated into the second polynucleotide strand; and detecting a signal from the second polynucleotide strand using a photodetector positioned adjacent the probe molecule.

Methods for analyzing the data collected from hybridization to arrays are well known in the art. In general, reactions occur in solution, e.g., a buffered solution. Typically, a solution is applied to the microarray substrate, and a reaction, including, but not limited to, hybridization (e.g., nucleic acid hybridization); an enzymatic reaction; a chemical reaction; protein-protein binding; protein-nucleic acid binding; and the like. Those skilled in the art can readily select appropriate reaction conditions, e.g., pH, temperature, ion concentration, etc., using standard protocol texts. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the test nucleic acids from the remaining data. The resulting data can be displayed as an image with the intensity in each region varying according to the binding affinity between associated oligonucleotides and/or polynucleotides and the test nucleic acids.

Oligonucleotides having a sequence unique to a particular target gene can be used in the present invention. Different methods may be employed to choose the specific region of the gene to be targeted. A rational design approach may also be employed to choose the optimal oligonucleotide sequence for the hybridization array. Preferably, the region of the gene that is selected is chosen based on the following criteria. First, the sequence that is chosen should yield an oligonucleotide composition that preferably does not cross-hybridize with any other oligonucleotide composition present on the array. Second, the sequence should be chosen such that the oligonucleotide composition has a low probability of cross-hybridizing with an oligonucleotide having a nucleotide sequence found in any other gene, whether or not the gene is to be represented on the array from the same species of origin, e.g., for a human array, the sequence will not be present in any other human genes. As such, sequences that are avoided include those found in: highly expressed gene products, structural RNAs, repeated sequences found in the sample to be tested with the array and sequences found in vectors. A further consideration is to select oligonucleotides with sequences that provide for minimal or no secondary structure, structure which allows for optimal hybridization but low non-specific binding, equal or similar thermal stabilities, and optimal hybridization characteristics.

As an example, a series of microarray spots are pipetted onto a microarray substrate. Each spot contains multiple copies of a polymer, wherein, in a given spot, the polymers are substantially identical to one another, e.g., wherein 98% or more, preferably 99% or more, of the copies of the polymer are identical to one another. Preferably, all copies (i.e., 100%) of the polymer within a microarray spot are identical to one another. As an example, the first in the series of microarray spots could contain a nucleic acid that specifically hybridizes to nucleic acid of a first pathogenic microorganism, the second in the series of microarray spots could contain a nucleic acid that specifically hybridizes to nucleic acid of a second pathogenic microorganism which is different from the first pathogenic microorganism, and so on. In this manner, a series of spots, each containing a nucleic acid that specifically hybridizes to a given pathogenic microorganism could be generated, which would provide a diagnostic tool to identify an unidentified pathogen in a biological sample.

A further example of an application is in dynamic allele-specific hybridization (DASH). Howell et al. (1999) Nat. Biotech. 17:87-88. A double-stranded polynucleotide specific intercalating dye such as ethidium bromide is included in the hybridization solution. Upon excitation, the dye will emit fluorescence in proportion to the amount of hybridized polynucleotides. Further, upon monitoring the excitation while increasing the temperature of the sample, a determination can be made as to the existence of a mis-match in the hybridized duplex, e.g. a duplex which contains a mis-match will have a lower melting temperature and therefore exhibit a decrease in fluorescence at a lower temperature than a perfectly matched duplex. This is useful, for example, in identifying alleles in DNA.

Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; 5,874,219; WO 95/21265; WO 96/31622; WO 97/10365; WO 97/27317; EP 373 203; and EP 785 280. References that disclose the synthesis of arrays and reagents for use with arrays include: Matteucci and Caruthers (1981) J. Am. Chem. Soc. 103:3185-3191; Beaucage and Caruthers (1981) Tetrahedron Letters 22(20):1859-1862; Adams et al. (1983) J. Am. Chem. Soc. 105:661-663; Sproat and Brown (1985) Nucleic Acids Research 13(8):2979-2987; Crea and Horn (1980) Nucleic Acids Research 8(10):2331-48; Andrus et al. (1988) Tetrahedron Letters 29(8):861-4; Applied Biosystems User Bulletin, Issue No. 43, Oct. 1, 1987, “Methyl phosphonamidite reagents and the synthesis and purification of methyl phosphonate analogs of DNA”; Miller et al. (1983) Nucleic Acids Research 11:6225-6242. Each of these is incorporated herein by reference as exemplary methods of use of microarray substrates of the present invention.

Exemplary Specific Embodiments of the Microarray Substrate of the Invention

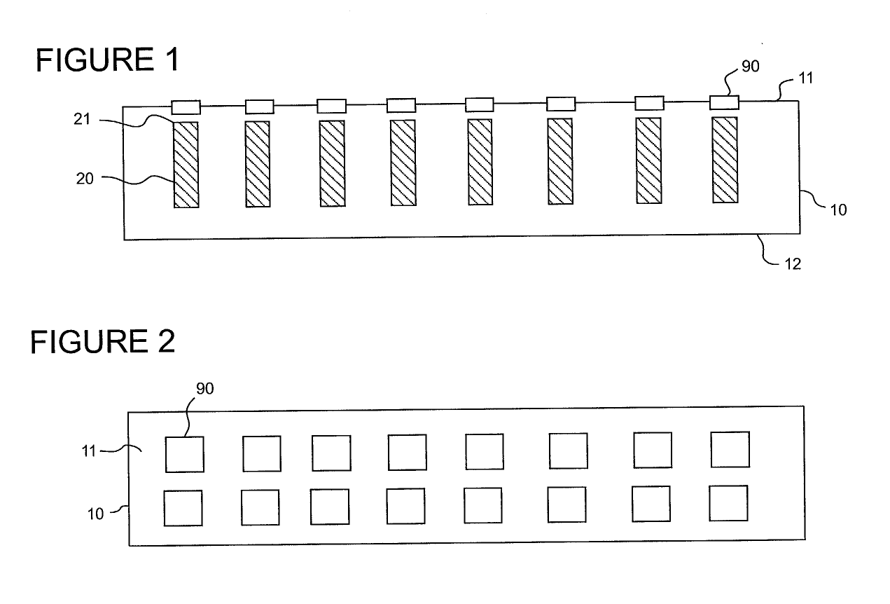

Referring generally to one non-limiting embodiment of the microarray substrate 5 of the invention is illustrated in FIGS. 1-3. FIG. 1 presents a cut-away view of solid substrate 10 which comprises a first planar surface 11 and a second planar surface 12. First planar surface 11 comprises a series of microarrays 90, each of which contains a plurality of substantially identical copies of a single probe or polymer. Directly beneath and integrated within the substrate 10 is a photodetector 20. Photodetector 20 comprises a first end 21 which is proximal to first planar surface 11 of solid substrate 10. FIG. 2 presents a perspective view of the first planar surface 11 of the solid substrate 10, showing multiple microarrays 90. Each microarray contains a plurality of identical copies of a single probe or polymer, which differs from one microarray to the next.

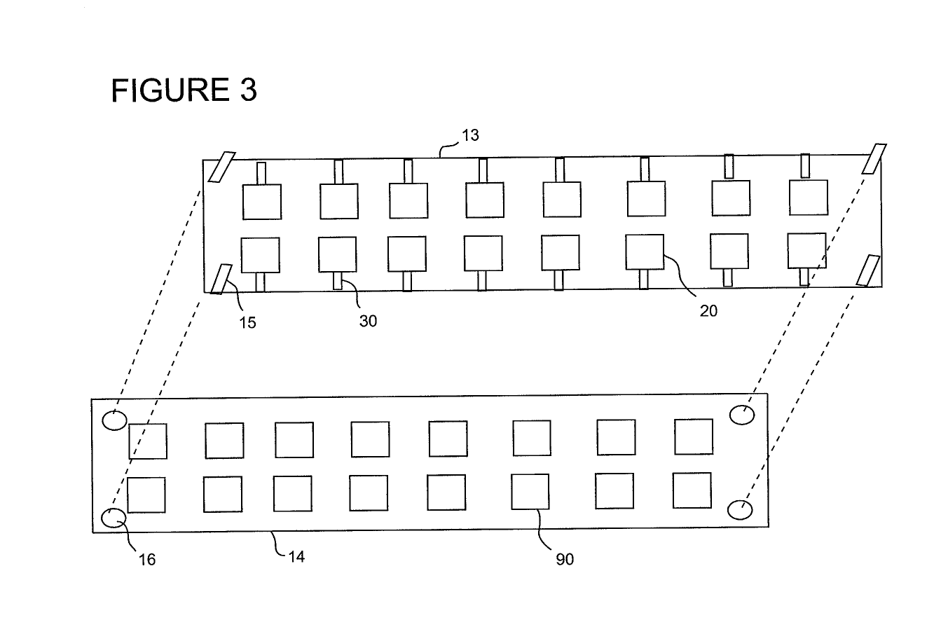

FIG. 3 depicts an exemplary embodiment of the invention in which the substrate 10 comprises a photodetector layer 13 comprising photodetectors 20, and a microarray, or polymer, layer 14 (comprising the microarray spots 90), wherein the photodetector substrate layer and polymer substrate layers are detachable from one another. Also shown in this view are signal transmission means 30 connected to each photodetector. In this exemplary embodiment, photodetector substrate layer 13 comprises pegs 15 extending upward, which are sized to fit into holes 16 in microarray substrate layer.

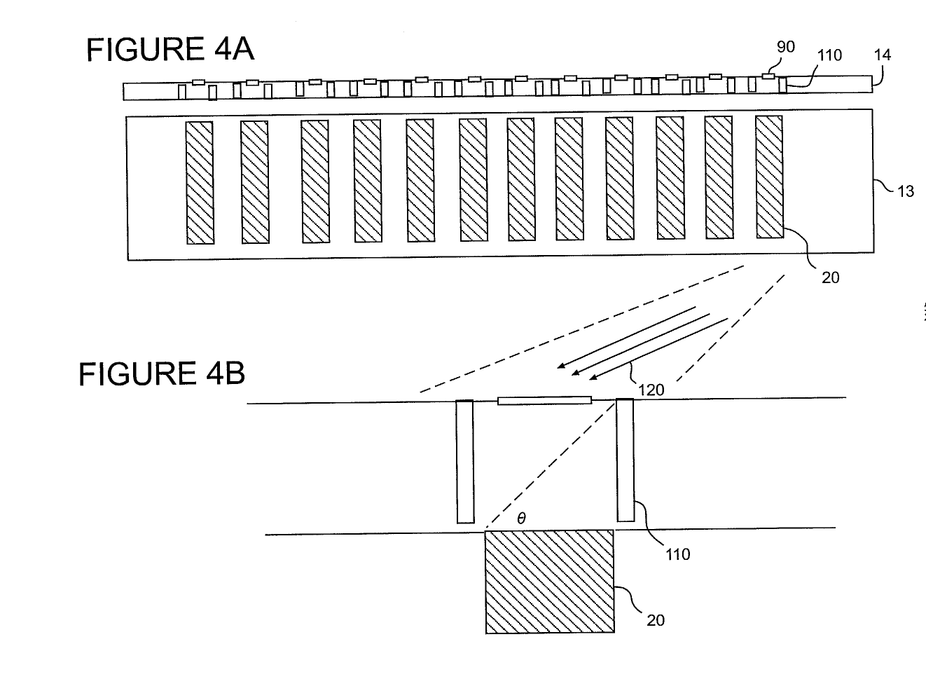

FIGS. 4A and 4B depict an exemplary embodiment of the invention comprising a radiant energy selection means. In this exemplary embodiment, as shown in FIGS. 4A and 4B, the angle of incident light is less than 90° to the plane of the photodetector layer 13, and louvers 110 have been deposited onto the surface of the photodetector layer, or, alternatively, into the microarray layer 14, and are embedded at least partially within a gap-filling layer, e.g., a glass or a polymer matrix. As shown in more detail in FIG. 4B, incident light 120 emitted from the laser source excites a fluorophore attached to a polymer in a microarray spot 90, which fluorophore emits radiant energy. Louvers 110 in the polymer layer 14 serve to reduce the amount of incident light that is detected by the photodetector 20.

Detection Devices for Use with Substrates of the Invention

The present invention further provides a detection device for use in conjunction with the substrates of the present invention. A detection device of the invention detects an electrical signal from a photodiode integrated into the microarray substrate. A detection device can comprise a component which converts the electrical signal into a digital signal, and can send the electrical signal (or a digitally converted form thereof) to a linked computer, which can store, manage, and process the information received.

A detection device of the invention comprises an element for immobilizing the microarray substrate; a reading device for reading an electronic signal from a signal transmission means of the substrate; and a microprocessor for storing, managing, and processing information provided by an electronic signal detected by the reading device. Data may also be presented as a digital readout. Methods and devices for converting a signal emitted from a photodiode into a digital signal are known in the art, and can be used in conjunction with the detection device of the invention. See, e.g., U.S. Pat. No. 4,990,765; and U.S. Pat. No. 5,850,195.

The device may further comprise a means for regulating the temperature within the detection device. Regulating the temperature may find use in applications in which association of complementary polymers is affected by temperature. As an example, stringency of nucleic acid hybridization is affected, in part, by temperature. As one non-limiting example, the temperature may be increased to 68° C. for stringent nucleic acid hybridization conditions. Nucleic acid hybridization conditions have been described in more detail hereinabove. Regulating the temperature may also find use in enzymatic reactions, where the temperature is adjusted to the temperature optimum of the enzyme being used. As an example, a reaction using an enzyme derived from an extreme thermophile can be carried out. A non-limiting example of such reactions is a polymerase chain reaction using a thermostable DNA polymerase (e.g., from Thermus aquaticus). Alternatively, the temperature can be adjusted so as to inactivate an enzyme, e.g., by raising the temperature well above the temperature optimum for an enzyme.

The means for regulating temperature can also be one that cools the device to temperatures below about −10° C., below about −20° C., or below about −30° C., down to about −40° C. Cooling the device to such low temperatures once the binding/hybridization reaction has already occurred confers the advantage of further reducing electrical noise, i.e., electrical signals from neighboring microarray spots, or other extraneous electrical signals.

Thus, a temperature regulator can regulate the temperature in a range of from about −40° C. to about 95° C., from about −30° C. to about 90° C., from about −20° C. to about 80° C., from about 10° C. to about 75° C., from about 0° C. to about 65° C., from about 4° C. to about 60° C., from about 10° C. to about 50° C., from about 17° C. to about 45° C., or from about 25° C. to about 30° C., or any selected temperature or temperature range within any of the foregoing ranges. In addition, a temperature regulator may provide for, e.g., a progressive increase or decrease in temperature over time, or may provide for a cycle(s) of two or three different temperatures (e.g., 95° C., 50° C., 72° C.).

In some embodiments, the detection device may further comprise a means for moving a (first) protective layer (e.g., a first polymer layer, comprising the polymers) away from the photodiode substrate layer, and exchanging it for a second polymer layer, which is different from the first polymer layer. As one non-limiting example, the means for moving the polymer layer may be an arm which comprises a means for grasping a polymer layer. The arm may be movably connected to a portion of the detection (reading) device.

A variety of radiant energy may be detected using the substrate and device of the invention. Suitable labels include, but are not limited to, radioisotopes; enzymes whose products are detectable (e.g., luciferase, β-galactosidase, and the like); fluorescent labels (e.g., fluorescein isothiocyanate, rhodamine, a fluorescent protein, phycoerythrin, and the like); a cyanine dye; fluorescence-emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. Examples of fluorescent labels include, but are not limited to, fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6carboxyrhodamine (TAMRA). Radioactive labels include, but are not limited to, ³²P, ³⁵S, ³H, and the like. The label may be a two-stage system, where the DNA or other polymer is conjugated to biotin, haptens, etc. having a high affinity binding partner, e.g. avidin, specific antibodies, etc., where the binding partner is conjugated to a detectable label.

A light source such as a light-emitting diode (LED) can be in the reading device in register with each microarray spot, each of which light sources can be addressable, allowing one to turn on each light source individually, in sequence, or in some pattern, such as even-odd-even-odd, thereby further reducing cross-talk. Alternatively, laser LEDs and vertical cavity surface emitting lasers (VCSELs) (Emcore, Somerset N.J.), can be used as the light source. Arrays of VCSELs have been described, and methods of making such arrays can be used in the present invention. U.S. Pat. No. 6,023,485.

The reading device may further comprise a means for varying the angle of incident light of a laser or other light source. A means for varying the angle of incident light finds use particularly when it is desired to avoid detection of the incident light by the photodetector, e.g., when a laser light source is used to excite a fluorophore, as described above.

In some embodiments, a light signal is generated without the need to irradiate the microarray. As an example, a target sequence may comprise a chromogenic substance emitting a light signal. In other embodiments, a radiant energy signal is generated upon irradiation of the microarray with excitation radiation. Application of excitation radiation is necessary when a target molecule comprises a fluorescent label. In these embodiments, the detection device comprises an excitation light source. Suitable excitation light sources for use in these embodiments are lasers including, but not limited to, argon lasers, diode lasers, helium neon lasers, dye lasers, Nd:YAG lasers, arc lamps, and the like. In some of these embodiments, the stage or body which holds the microarray substrate may also serve as an x-y translation table to allow movement of the microarray substrate such that different microarray spots or regions can be irradiated.

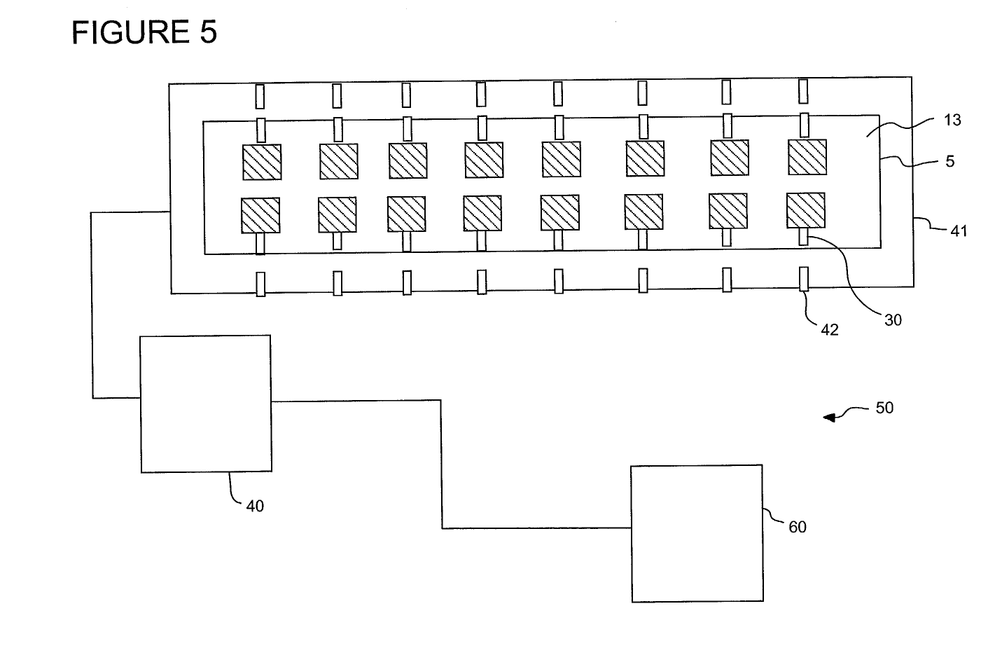

FIG. 5 presents a view of an exemplary embodiment of a detection device. Detection device 50 comprises a reading device 40 which comprises a stage 41 for holding microarray substrate 5 (shown in this view is photodetector layer 13, without polymer layer 14), and electrical contacts 42 which contact signal transmission means 30 and provide for transmission of an electrical signal from the signal transmission means to the reading device. Detection device 50 further comprises a microprocessor 60 which is electrically coupled to reading device 40. Microprocessor 60 stores, manages, and processes data received from the reading device.

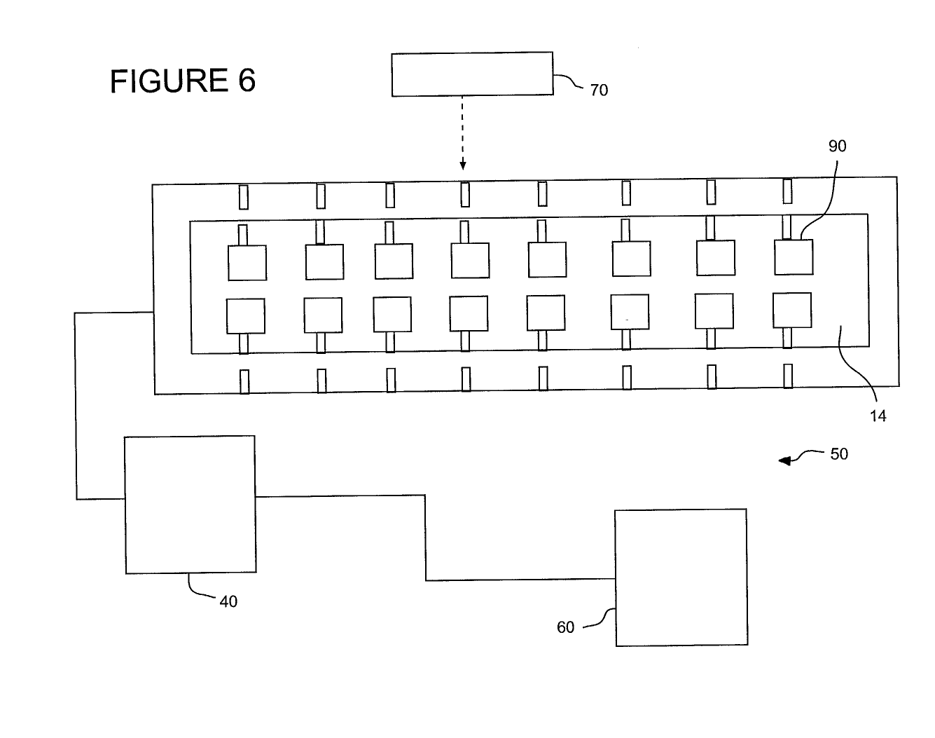

FIG. 6 presents a view of an exemplary embodiment of a detection device 50 essentially as in FIG. 5, which further comprises an excitation radiation source 70. Shown in this view is polymer layer 14 which is on top of photodetector layer 13. Irradiation of polymers in microarray substrate 10, which polymers may be bound to (e.g., hybridized to) a target polymer labeled with, e.g., a fluorophore, results in emission of radiant energy from the target sequence comprising the fluorescent label.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A device comprising:
a solid substrate which provides a surface;
a plurality of different probe polymer sequences bound to the surface, wherein each different sequence is bound to a distinct area of the surface;
a plurality of photodetectors positioned in a manner such that a signal emitted from a distinct area of probe polymer sequences can be detected and differentiated from a signal of another distinct area of probe polymer sequences.

2. The device of claim 1, wherein the photodetector is selected from the group consisting of a photodiode, a charge-coupled device, a photoconductive cell, an avalanche photodiode, a photoresistor, a photoswitch, a phototransistor, a phototube, a photovoltaic cell, and a light-to-frequency converter.

3. The device of claim 1, wherein the photodetector is a photodiode.

4. The device of claim 3, wherein each photodiode is associated with a single distinct area.

5. The device of claim 1, wherein the substrate comprises a first layer and a second layer, wherein the first layer is detachably positioned on the second layer, wherein the first layer provides a surface to which a plurality of different probe polymer sequences are bound, wherein each different polymer sequence is bound to a distinct area of the surface, and wherein the second layer comprises a plurality of photodetectors positioned in a manner such that a signal emitted from a distinct area of probe polymer sequences on the first layer can be detected and differentiated from a signal of another distinct area of probe polymer sequences.

6. The device of claim 1, wherein the probe polymers are selected from the group consisting of single-stranded naturally-occurring nucleotide sequences, single-stranded modified nucleotide sequences, single-stranded synthetic nucleotide sequences, and single-stranded semi-synthetic nucleotide sequences.

7. The device of claim 1, further comprising a signal transmission means connected to the plurality of photodetectors.

8. The device of claim 7, further comprising a means for analyzing signals received from the signal transmission means.

9. The device of claim 1, wherein said substrate comprises a substance selected from the group consisting of silicon, GaAs, SiO₂, glass, and functionalized glass.

10. The device of claim 1, wherein at least one of said photodetectors comprises positional address information.

11. The device of claim 1, further comprising a means for regulating the temperature integrated within said solid substrate.

12. The device of claim 8; further comprising a microprocessor for storing, managing, and processing information provided by an electronic signals received.

13. The device of claim 12, further comprising a light source; and a means for directing the light source.

14. The device of claim 12, further comprising an the immobilizing element in the form of an x-y translation table.

15. The device of claim 1, further comprising a means for regulating the temperature within the device.

16. The device of claim 1, further comprising a radiant energy selection means.

17. The device of claim 16, wherein the radiant energy selection means is selected from the group consisting of an interference filter layer, an optical wave guide, a polarization layer, a time-resolved fluorescence means, and a grating.

18. A method of detecting a probe molecule in a microarray, comprising
a) allowing a labeled target molecule to hybridize to a probe molecule bound to a substrate, forming a probe-target hybrid; and
b) detecting a signal from the probe-target hybrid using a photodetector positioned adjacent the probe molecule.

19. The method of claim 18, wherein the substrate is comprised of a plurality of distinct areas which each have bound thereto probe molecules and further wherein a plurality of photodetectors are positioned adjacent the distinct areas in a manner allowing for differentiating among signals received from the distinct areas.

20. The method of claim 18, further comprising analyzing signals received from the photodetectors.

21. A method of detecting a probe molecule in a microarray, comprising:
a) allowing an oligonucleotide primer molecule to hybridize to a probe molecule bound to a substrate, forming a probe-primer hybrid;
b) contacting the probe-primer hybrid with a DNA polymerase, forming a reaction mixture, under conditions that promote addition of a nucleotide to the 3′ end of the primer, such that a second polynucleotide strand is generated that comprises a nucleotide sequence complementary to the probe sequence such the second polynucleotide strand hybridizes to the probe, forming a probe-second polynucleotide strand hybrid, wherein the reaction mixture comprises a labeled nucleotide, and wherein the labeled nucleotide is incorporated into the second polynucleotide strand; and
c) detecting a signal from the second polynucleotide strand using a photodetector positioned adjacent the probe molecule,
wherein the substrate is comprised of a plurality of distinct areas which each have bound thereto probe molecules and further wherein a plurality of photodetectors are positioned adjacent the distinct areas in a manner allowing for differentiating among signals received from the distinct areas.
